(19) United States
(12) Patent Application Publication
(10) Pub. No.: US 2025/0342962 A1
(43) Pub. Date: Nov. 6, 2025

(54) METHOD AND MONITORING SYSTEM USING MACHINE LEARNING TO MONITOR COGNITIVE OR PHYSICAL IMPAIRMENT

(71) Applicant: 12163004 CANADA INC, MONTREAL (CA)

(72) Inventor: Jean-Francois POISSON, MONTREAL (CA)

(73) Assignee: 12163004 CANADA INC, MONTREAL (CA)

(21) Appl. No.: 19/196,605

(22) Filed: May 1, 2025

Related U.S. Application Data
(60) Provisional application No. 63/642,456, filed on May 3, 2024.

(51) Int. Cl. G16H 50/20; G16H 20/00; G16H 40/67

(52) U.S. Cl. CPC G16H 50/20; G16H 20/00; G16H 40/67

(57) ABSTRACT

Method and monitoring system using machine learning to monitor cognitive or physical impairment. The monitoring system receives data from a plurality of devices (e.g. a sensing device, a personal electronic device, a smart appliance) located in a living environment of a monitored person and generates monitoring data based on the received data. The monitoring system executes a machine learning algorithm, the machine learning algorithm using a predictive model to determine one or more outputs based at least on the monitoring data. The one or more outputs comprise at least one of a cognitive impairment indicator (indicative of whether the monitored person is affected by cognitive impairment) and a physical impairment indicator (indicative of whether the monitored person is affected by physical impairment). Optionally, a determination is made based on at least one of the indicators to activate one or more functionalities of an assistance software.

### TECHNICAL FIELD

The present disclosure relates to the field of elderly care assistance. More specifically, the present disclosure presents a method and monitoring system using machine learning to monitor cognitive or physical impairment.

### BACKGROUND

Most countries are now facing the challenge of supporting an ageing population. For people who have spent a large part of their life in a house or an apartment, it is very difficult when they age to abandon their housing, to live in a retirement/nursing home.

This is especially difficult for people living alone, to maintain their autonomous lifestyle in a housing of their own. However, it is also considered beneficial from a physical, mental and cognitive perspective to remain autonomous as long as possible.

Various technological solutions are being developed to facilitate the everyday life of people living alone in their housing. For example, these solutions aim at facilitating physical tasks, facilitating intellectual tasks, providing entertaining and challenging activities (physically and intellectually), etc.

One important aspect is to have the capability to detect a decline in the cognitive and/or physical capabilities of a person. However, it is not easy to detect this decline since the person is living alone and may have limited/sporadic interactions with other persons.

The development of technologies in the field of connected homes provides solutions for monitoring different aspects of the file of a person. However, the interpretation of the monitoring data (collected for example by various types of sensors deployed in a living environment), for the purpose of detecting a decline in the cognitive and/or physical capabilities of a person is currently a technical challenge.

There is therefore a need for a new method and a monitoring system using machine learning to monitor cognitive or physical impairment.

### SUMMARY

According to a first aspect, the present disclosure relates to a method using machine learning to monitor cognitive or physical impairment. The method comprises receiving, by a processing unit of a monitoring system, data from a plurality of devices located in a living environment of a monitored person. The method comprises generating, by the processing unit, monitoring data based on the received data. The method comprises executing, by the processing unit, a machine learning algorithm. The machine learning algorithm uses a predictive model to determine one or more outputs based at least on the monitoring data. The one or more outputs comprise at least one of a cognitive impairment indicator and a physical impairment indicator. The cognitive impairment indicator indicates whether the monitored person is affected by cognitive impairment. The physical impairment indicator indicates whether the monitored person is affected by physical impairment.

According to a second aspect, the present disclosure relates to non-transitory computer readable medium comprising instructions executable by a processing unit of a monitoring system. The execution of the instructions by the processing unit of the monitoring system provides for using machine learning to monitor cognitive or physical impairment by implementing the aforementioned method.

According to a third aspect, the present disclosure relates to a monitoring system. The monitoring system comprises at least one communication interface, memory storing a predictive model, and a processing unit. The processing unit receives data from a plurality of devices located in a living environment of a monitored person. The processing unit generates monitoring data based on the received data. The processing unit executes a machine learning algorithm. The machine learning algorithm uses a predictive model to determine one or more outputs based at least on the monitoring data. The one or more outputs comprise at least one of a cognitive impairment indicator and a physical impairment indicator. The cognitive impairment indicator indicates whether the monitored person is affected by cognitive impairment. The physical impairment indicator indicates whether the monitored person is affected by physical impairment.

In a particular aspect, the one or more outputs of the machine learning algorithm comprises the cognitive impairment indicator. In a particular embodiment, the cognitive impairment indicator is transmitted to a third party device. In another particular embodiment, a determination is made based at least on the cognitive impairment indicator that one or more functionalities of an assistance software need to be activated, the assistance software providing assistance in the daily life of the monitored person.

In another particular aspect, the one or more outputs of the machine learning algorithm comprises the physical impairment indicator. In a particular embodiment, the physical impairment indicator is transmitted to a third party device. In another particular embodiment, a determination is made based at least on the physical impairment indicator that one or more functionalities of an assistance software need to be activated, the assistance software providing assistance in the daily life of the monitored person.

In still another particular aspect, the plurality of devices located in the living environment of the monitored person comprise at least one of the following: a sensing device, a personal electronic device and a smart appliance.

In yet another particular aspect, the monitoring data comprise at least one of the following: an occurrence of an activity performed by the monitored person, a duration of an activity performed by the monitored person, a number of occurrences of an activity performed by the monitored person, an occurrence of an interaction of the monitored person with a device or an object located in the living environment of the monitored person, a duration of an interaction of the monitored person with a device or an object located in the living environment of the monitored person, a number of occurrences of an interaction of the monitored person with a device or an object located in the living environment of the monitored person, an occurrence of a fall of the monitored person, a number of occurrences of a fall of the monitored person, an average speed of the monitored person when walking in the living environment, an average time spent in an area, a maximum time spent in an area, a minimum time spent in an area, a number of visits to an area, a sleep quality metric, a health metric, a variation in the value of a metric generated based on the received data, information related to at least one of physical and cognitive capabilities of the monitored person, personal information related to the monitored person.

In another particular aspect, the machine learning algorithm implements a neural network, the predictive model comprising weights of the neural network.

### BRIEF DESCRIPTION OF THE DRAWINGS

Embodiments of the disclosure will be described by way of example only with reference to the accompanying drawings, in which:

FIG. 1 represents a monitoring system for performing cognitive or physical impairment monitoring;

FIG. 2 represents an alternative implementation of the monitoring system illustrated in FIG. 1;

FIG. 3 represents a method using machine learning to monitor cognitive or physical impairment;

FIG. 4 represents a machine learning algorithm used for determining a cognitive impairment indicator;

FIG. 5 represents a machine learning algorithm used for determining a physical impairment indicator;

FIG. 6 represents a machine learning algorithm used for simultaneously determining a cognitive impairment indicator and a physical impairment indicator;

FIG. 7 represents an assistance software having functionalities activated based on impairment indicator(s) detected by the machine learning algorithms of FIGS. 4, 5 and 6; and

FIG. 8 represents a neural network implementing the machine learning algorithm illustrated in FIG. 4.

### DETAILED DESCRIPTION

The foregoing and other features will become more apparent upon reading of the following non-restrictive description of illustrative embodiments thereof, given by way of example only with reference to the accompanying drawings. Like numerals represent like features on the various drawings.

Various aspects of the present disclosure generally address the need to provide a solution for detecting a decrease of cognitive and/or physical capabilities of an elderly person living in an apartment or a house. More specifically, the present disclosure describes a solution relying on a machine learning algorithm trained to process monitoring data collected in the living environment of the person, to detect cognitive or physical impairment of the person.

Throughout the present description, the expression cognitive impairment and cognitive impairment indicator may be interchanged with cognitive decline and cognitive decline indicator. Furthermore, the expressions physical impairment and physical impairment indicator may be interchanged with physical decline and physical decline indicator.

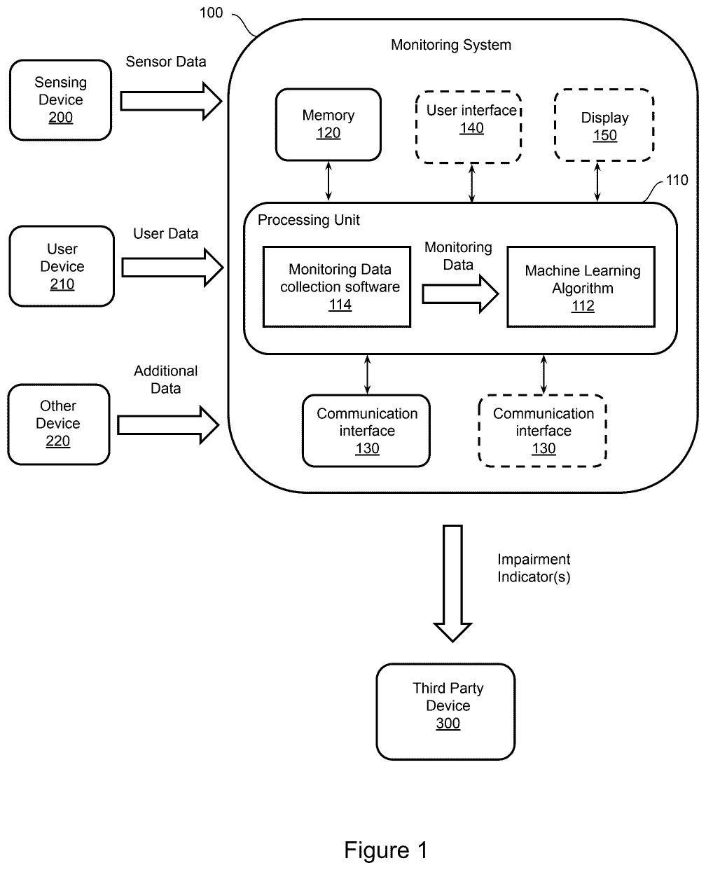

Referring now to FIG. 1, a monitoring system for performing cognitive or physical impairment monitoring is represented. A person, referred to as the monitored person, is monitored by the system. The monitoring system is adapted to monitor an elderly person, but can also be used to monitor other types of persons (e.g. an handicapped person). The present monitoring system is designed so as to rely on non-intrusive monitoring options, and correlates multiple sources of information to analyze activities, movements and sounds in a living environment of a monitored person, while providing privacy to the monitored person. Furthermore, by relying on multiple types of devices and machine learning algorithm, the present monitoring system adapts to each monitored person, identifies signs of cognitive and/or physical decline, and informs family or caregiver of the early signs of decline. Early detection of decline, while respecting privacy of the monitored person are key to assisting the monitored persons who still live at home, while guiding the family or caregiver on the needs of the monitored person.

The monitoring system 100 is adapted for communicating wirelessly or by wires with multiple types of devices 200, 210 and 220 capable of collecting data related to a person. The devices 200, 210 and 220 are deployed in the living environment of the person, such as an apartment or a house where the person is living. The devices 200, 210 and 220 may be deployed in a single room or different rooms of the living environment. The data collected by the different devices 200, 210 and 220 are transmitted to the monitoring system 100, which may also be located in the living environment, in a building where the devices 200, 210 and 220 are located, or remotely located.

A first type of devices consists of one or more sensing devices 200 deployed in the living environment. Each sensing device 200 generates sensor data, which are transmitted to the monitoring system 100.

An example of sensing device 200 includes a sound sensor capable of capturing a sound sequence generated by the person (e.g. when speaking, yelling, crying, laughing, etc.) or generated by an interaction of the person with its environment (e.g. something falling on the floor, an object being broken, etc.). Another example of sensing device 200 includes an image sensor capable of capturing a single image or a video sequence (e.g. an infrared image sensor, a visible image sensor, etc.).

Another example of sensing device 200 includes a radar capable of capturing data related to the movement of the person (e.g. a succession of positions of the person, a succession of movements, a speed of the person, an acceleration of the person, a decrease of the speed of movement of the monitored person, etc.). An analysis of the data transmitted by the radar can be performed by the monitoring system 100, for example to detect a fall of the person. Another example of sensing device 200 includes a presence detector capable of determining whether the person is present or not in an area (e.g. in a room). Another example of sensing device 200 includes a bed sensor (a sensor integrated to the bed) capable of collecting data related to a quality of the sleep of the person.

A person skilled in the art will readily understand that other types of sensing devices 200 may be deployed, to capture other types of data related to the person. Furthermore, any combination of various types of sensing devices may be used, the deployment of the various types of sensing devices in the living environment following various deployment configurations. A detailed description of the sensing devices 200 will not be provided, since the operation of sensing devices is well known in the art.

A second type of devices consists of one or more user devices 210 deployed in the living environment. A user device 210 is a device with which the person interacts, the interaction generating user data which are transmitted to the monitoring system 100.

A first category of user devices 210 consists of personal electronic devices, such as a smartphone, a tablet, a computer, a wearable device (e.g. a smartwatch, etc.), a television set, an electronic book reader, etc. Each of these personal electronic devices executes an embedded software capable of generating the user data transmitted to the monitoring system 100. Examples of user data include the time spent interacting with the device, the start time and end time of an interaction, the volume of the sound for a device generating sound, a type of activity performed on the device (e.g. reading, watching a video content or playing a game on a smartphone or tablet), a type of content being consumed (e.g. the type of program being played on a television set), sleep quality data and/or health data (e.g. heart rate, blood pressure, oxygen saturation, movements, etc.) monitored by a wearable device such as a smartwatch, etc.

A second category of user devices 210 consists of smart appliances, such as a smart fridge, a smart stove, a smart coffee maker, a smart kettle, etc. A smart appliance also executes an embedded software capable of generating the user data transmitted to the monitoring system 100. Examples of user data include data related during interaction with the appliance (e.g. determined over a given period of time), such as for example interactions. Examples of interactions include the number of times the person opens the fridge during a day, the number of times the person uses an appliance (e.g. stove, coffee maker, smart kettle, etc.) during a day, the time of each occurrence of the opening of the fridge or each occurrence of the usage of the stove (or coffee maker, or kettle), etc.

A person skilled in the art will readily understand that other types of user devices 210 may be used, to capture other types of user data related to the person. Furthermore, any combination of various types of user devices may be used. A detailed description of the user devices 210 will not be provided, as operation of such user devices are well known in the art.

The third type of devices, the other devices 220 may generate additional data, which is transmitted to the monitoring system 100. For example, the other device 220 may consist of personal tracking device(s) (e.g. smartphone, tablet, computer, smartwatch, etc.). The other devices 220 may further consist of devices used by individuals interacting with the monitored person, those individuals having a good understanding of the situation of the monitored person and providing observations and feedback to the monitoring system 100. Such individuals include for example close family and/or friends, health workers, etc. The other device 220 may or may not be located in the living environment of the monitored person, when transmitting the additional data.

A first exemplary type of additional data includes a monitoring survey generated on a regular basis (e.g. weekly, monthly, etc.). The monitoring survey is generated by the individual through the other device 220 and transmitted to the monitoring system 100. The monitoring survey comprises information related to (e.g. ratings of) at least one of physical capabilities and cognitive capabilities of the person being monitored. A second exemplary type of additional data includes a form providing personal information related to the person being monitored. The personal information includes a profile of the person (e.g. demographic information, information about the environment where the person is living, information related to the health of the person, information related to a medical condition of the person, information related to medications taken by the person, etc.). Alternatively or complementarily, the personal information includes preferences of the person (e.g. culinary preferences, favorite activities, items that the person likes or does not like, etc.).

Following is a detailed description of the components of the monitoring system 100. The monitoring system 100 comprises a processing unit 110, memory 120, at least one communication interface 130. The monitoring system 100 may comprise additional components, such as a user interface 140, a display 150, etc.

The processing unit 110 comprises one or more processors (not represented in FIG. 1) capable of executing instructions of a computer program. Each processor may further comprise one or several cores. The processing unit 110 executes a first software (computer program) implementing a machine learning algorithm 112. The processing unit 110 executes a second software (computer program) implementing a monitoring data collection functionality 114.

The memory 120 stores instructions of computer program(s) executed by the processing unit 110 (e.g. instructions for implementing the software 112 and 114), data generated by the execution of the computer program(s), data received via the communication interface(s) 130, etc. Only a single memory 120 is represented in FIG. 1, but the monitoring system 100 may comprise several types of memories, including volatile memory (such as a volatile Random Access Memory (RAM), etc.) and non-volatile memory (such as electrically-erasable programmable read-only memory (EEPROM), flash, etc.).

Each communication interface 130 allows the monitoring system 100 to exchange data with other devices (e.g. the sensing device(s) 200, the user device(s) 210, the other device(s) 220, a third party device 300, etc.) over a communication network (not represented in FIG. 1 for simplification purposes). For example, the communication network is a wired communication network, such as an Ethernet network; and the communication interface 130 is adapted to support communication protocols used to exchange data over the Ethernet network. Other types of wired communication networks may also be supported by the communication interface 130. In another example, the communication network is a wireless communication network, such as a Wi-Fi network; and the communication interface 130 is adapted to support communication protocols used to exchange data over the Wi-Fi network. Other types of wireless communication network may also be supported by the communication interface 130, such as a wireless mesh network, Bluetooth®, Bluetooth® Low Energy (BLE), etc. Each communication interface 130 usually comprises a combination of hardware and software executed by the hardware, for implementing the communication functionalities of the communication interface 130.

In a first exemplary implementation, the monitoring system 100 is a standard monitoring system (e.g. a tablet, a computer, a smartphone, a television, a cable box for a television, etc.). The standard monitoring system is configured to implement the monitoring functionalities described in the present disclosure. For instance, the machine learning algorithm 112 and the monitoring data collection software 114 are installed on the standard monitoring system, and the execution of the software 112 and 114 by the standard monitoring system provide the aforementioned monitoring functionalities. The standard monitoring system may be used by the person being monitored for other purposes (e.g. entertainment, reading, etc.).

In a second exemplary implementation, the monitoring system 100 is a dedicated monitoring system implementing the monitoring functionalities described in the present disclosure. For example, the dedicated monitoring system is similar to a set-top box. In this case, the dedicated monitoring system is not used by the person being monitored for other purposes. Furthermore, the dedicated monitoring system can be always on, allowing reception of data from the devices 200, 210 and 220 at any time.

A detailed description of the components of the sensing device 200, user device 210 and other device 220 is not represented in FIG. 1 for simplification purposes. The devices 200, 210 and 220 include a processing unit similar to the processing unit 110 of the monitoring system 100, for generating the data transmitted to the monitoring system 100. The devices 200, 210 and 220 also include a communication interface similar to the communication interface 130 of the monitoring system 100, for transmitting the generated data to the monitoring system 100.

Following is a detailed description of the software 114 and 112. The monitoring data collection software 114 receives the data transmitted by at least some of the sensing device(s) 200, user device(s) 210 and other device(s) 220 via the communication interface 130 of the monitoring system unit 100. Based on the type of received data, the data are directly used by the machine learning algorithm 112 or processed by the collection software 114 before being used by the machine learning algorithm 112.

To generalize, the monitoring data collection software 114 generates monitoring data used by the machine learning algorithm 112, based on the data received from the sensing device(s) 200, the user device(s) 210 and the other device(s) 220. The generation of the monitoring data comprises either processing the received data or directly using the received data.

Sensor data received from the sensing devices 200 generally need to be processed, to generate monitoring data which are used by the machine learning algorithm 112. Following are examples of monitoring data based on the types of sensors being deployed.

In the case of images or videos generated by an image sensor, an algorithm (e.g. a machine learning algorithm specialized in image processing) is used to extract useful features (monitoring data used by the machine learning algorithm 112), such as identifying an activity performed by the person, identifying an unusual event in relation to the person (e.g. sleeping during the day, falling, dropping an object, breaking an object, etc.), etc.

In the case of a radar, an algorithm is used to extract meaningful information from the radar-generated image(s), such as identifying the monitored person from dots, movement of the monitored person from a sequence of radar-generated images, fall of the monitored person, or any other type of information which may be extracted from the radar-generated image(s).

In the case of sound generated by a sound sensor, an algorithm (e.g. a machine learning algorithm specialized in sound processing) is also used to extract useful features (monitoring data used by the machine learning algorithm 112). The extracted features are similar to the previously described features for an image sensor, e.g. one particular audio signal or a sequence of audio signals

The analysis of the received images and/or sounds can also be used to determine the following monitoring data: if the person is speaking (possibly alone or with another person), yelling, crying, laughing, etc. The occurrence of one of these events (or the number of occurrences of one of these events over a given period of time) is used by the machine learning algorithm 112.

In the case of data generated by a radar, an algorithm (e.g. a machine learning algorithm specialized in fall detection) is used to determine whether a fall of the person has occurred or not. The occurrence of a fall (or the number of occurrences of a fall over a given period of time) is used by the machine learning algorithm 112. Another example of a metric calculated based on the data generated by the radar is an average speed of the monitored person when walking in the living environment.

In the case of data generated by a presence detector sensor, the following metrics can be calculated (e.g. over a given period of time) and used by the machine learning algorithm 112: average time spent in an area (e.g. a room), maximum time spent in an area (e.g. a room), minimum time spent in an area (e.g. a room), number of visits to an area (e.g. a room), etc.

In the case of data collected by bed sensor(s), sleep quality metrics are determined based on the collected data and used by the machine learning algorithm 112 (e.g. sleep duration, number of awakenings, etc.). For example, pressure sensors integrated into the bed are used to determine the sleep duration and the number of awakenings. Alternatively or complementarity, parameters representative of sleep quality (e.g. heart rate, movements, etc.) are collected by a user device (e.g. a smartwatch), to determine the sleep quality metrics.

User data received from the user devices 210 (e.g. personal electronic device or smart appliance) also generally need to be processed, to generate monitoring data which are used by the machine learning algorithm 112. Examples of monitoring data (e.g. determined over a given period time) include: time spent interacting with a user device, number of interactions with the user device, time spent performing a given type of activity supported by the user device (duration of the activity), number of occurrences of the given type of activity supported by the user device, dedicated metrics related to a particular type of user device (as described previously, such as average sound volume of a device generating sound), sleep quality metrics as mentioned previously, health metrics (e.g. heart rate, blood pressure, oxygen saturation, etc.), etc.

Furthermore, a variation in the value of a metric generated based on the received data can also be determined and used as input of the machine learning algorithm 112. For example, a variation in the duration of an event, activity, etc. In another example, a variation in the number of occurrences of an event, activity, etc.

The information received from the other device(s) 220 (e.g. in a monitoring survey or through any other electronic medium such as email, texts, etc.) is used directly by the machine learning algorithm 112. Alternatively, the information is converted into a format adapted to be processed by the machine learning algorithm 112 (e.g. conversion of an alphanumeric entry into a discrete numeric value).

Following are examples of physical capabilities and cognitive capabilities related data which may be received by the monitoring system 100: data related to the lifting of an object of a certain weight, data related to bending capability, data related to standing up capability, data related to awakeness period(s), data related to sleep (e.g. number of consecutive hours, naps, sleep and waking habits), data related to performing of pre-defined manual task(s), data related to capability to perform pre-defined activities requiring cognitive capabilities (e.g. reading, playing a game, socializing, watching or listening to audio or video content, etc.), etc. The data for cognitive capabilities can further be rated for difficulty and duration.

Following are examples of personal information (defining a profile of the monitored person) received in a personal information form: age, sex, level of autonomy, lives alone or not, lives in a house or an apartment, lives at the ground floor or not, needs to perform maintenance/gardening or not, cooks or not, owns a car or not, does the grocery shopping or not, owns a cellular phone or not, owns a tablet or not, owns a computer or not, number of children, number of caregivers, has a specific medical condition or not (e.g. Alzheimer, allergies, wheel chair, walker, etc.), etc.

Following are examples of personal information (defining preferences of the monitored person) received in a personal information form: favorite color(s), favorite food, favorite meal(s), favorite music, favorite artist(s), favorite type of humor, favorite animal(s), favorite scent(s), etc. The preferences can also be defined in a negative way, by stating items the monitored person does not like.

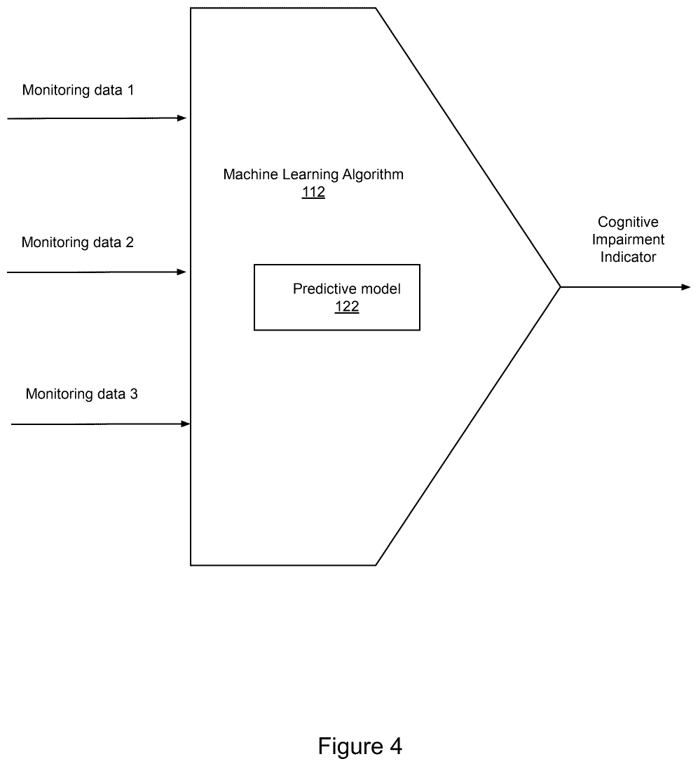

Reference is now made concurrently to FIGS. 1, 4, 5 and 6, where FIGS. 4, 5 and 6 represent several implementations of the machine learning algorithm 112.

The machine learning algorithm 112 uses a predictive model 122 to generate one or more outputs based on inputs. The predictive model 122 is generated during a training phase and stored in the memory 120 of the monitoring system 100. For example, the predictive model is generated and transmitted by a training server (not represented in the Figures for simplification purposes); and received via the communication interface 130 of the monitoring system 100.

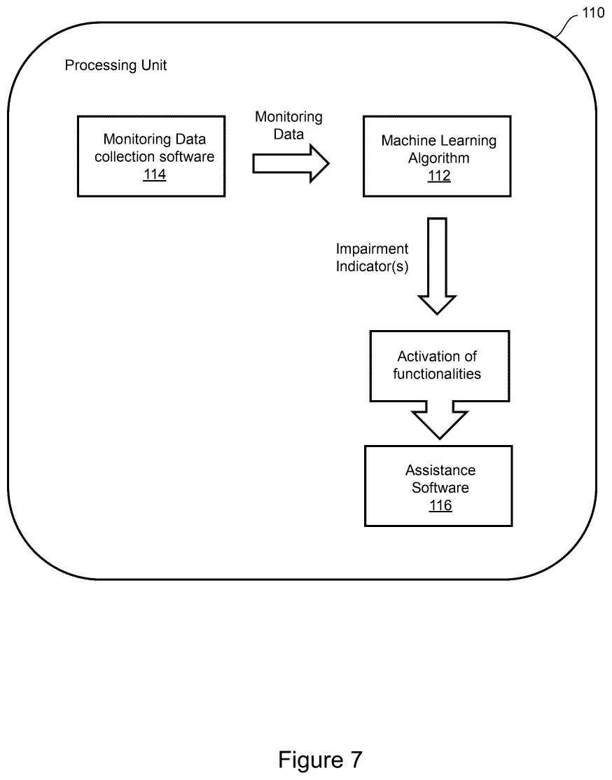

Several types of machine learning algorithms may be used in the context of the present disclosure, such as (without limitations) a neural network, linear regression, logistic regression, decision tree, support vector machine (SVM) algorithm, K-nearest neighbors algorithm, K-means algorithm, random forest algorithm, etc. The implementation of the machine learning algorithm by a neural network will be detailed later in the description, in relation to FIG. 7.

The inputs of the machine learning algorithm 112 are the monitoring data generated by the monitoring data collection software 114, based on the data received from at least one the devices 200, 210 and 220. Any combination of the previously mentioned types of monitoring data may be used as inputs. Furthermore, for a given type of monitoring data, a single instance of the monitoring data is used as input. Alternatively, a series of consecutive instances of the monitoring data are used as inputs.

For illustration purposes only, FIG. 4 represents three different types of monitoring data being used as inputs. However, any number of inputs greater than one may be used. Furthermore, monitoring data 1 may consist of a single instance or a series of consecutive instances of the same type of monitoring data (the same applies to monitoring data 2 and 3).

FIG. 4 represents a first implementation with one output: a cognitive impairment indicator. The indicator is an indication of whether the monitored person is affected by cognitive impairment, based on the processing of the inputs by the machine learning algorithm 112. For example, the cognitive impairment is representative of at least one of the following: difficulty to perform one or more intellectually demanding tasks (e.g. reading, counting, having a conversation, etc.), difficulty to concentrate, memory problems, etc.

For example, the indicator is a Boolean representative of the cognitive impairment (e.g. true if a cognitive impairment is determined and negative if no cognitive impairment is determined). In another exemplary implementation, the indicator is a probability of occurrence of the cognitive impairment (e.g. a percentage of chances that the cognitive impairment is occurring or alternatively a percentage of chances that the cognitive impairment is not occurring). In still another exemplary implementation, the indicator is one among a pre-defined set of values representatives of different levels of cognitive impairment (e.g. no cognitive impairment, light cognitive impairment, strong cognitive impairment, critical cognitive impairment, etc.). A person skilled in the art would readily understand that other implementations of the cognitive impairment indicator may be used.

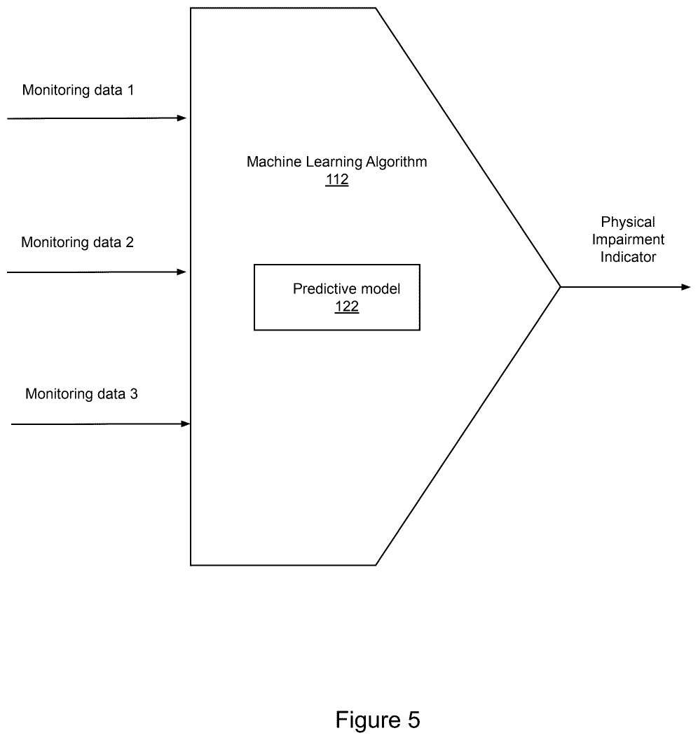

FIG. 5 represents a second implementation with one output: a physical impairment indicator. The indicator is an indication of whether the monitored person is affected by physical impairment, based on the processing of the inputs by the machine learning algorithm 112. For example, the physical impairment is representative of at least one of the following: difficulty to perform one or more physically demanding tasks (e.g. walking, carrying an object, standing up, etc.), general state of tiredness, difficulty to move use a part of the body (e.g. moving an arm, turning the head, etc.), etc.

As mentioned previously, the indicator is one of the following: a Boolean, a probability of occurrence, one among a pre-defined set of values representative of different levels of physical impairment or decline, one among a pre-defined set of values representative of different levels of cognitive impairment or decline, etc.

For illustration purposes only, FIG. 5 represents three different types of monitoring data being used as inputs. However, any number of inputs greater than one may be used. Furthermore, monitoring data 1 may consist of a single instance or a series of consecutive instances of the same type of monitoring data (the same applies to monitoring data 2 and 3).

The types of monitoring data used as inputs for the implementations illustrated in FIGS. 4 and 5 may be entirely different, or some of them may be common. However, the predictive models 122 used for the implementations illustrated in FIGS. 4 and 5 are different.

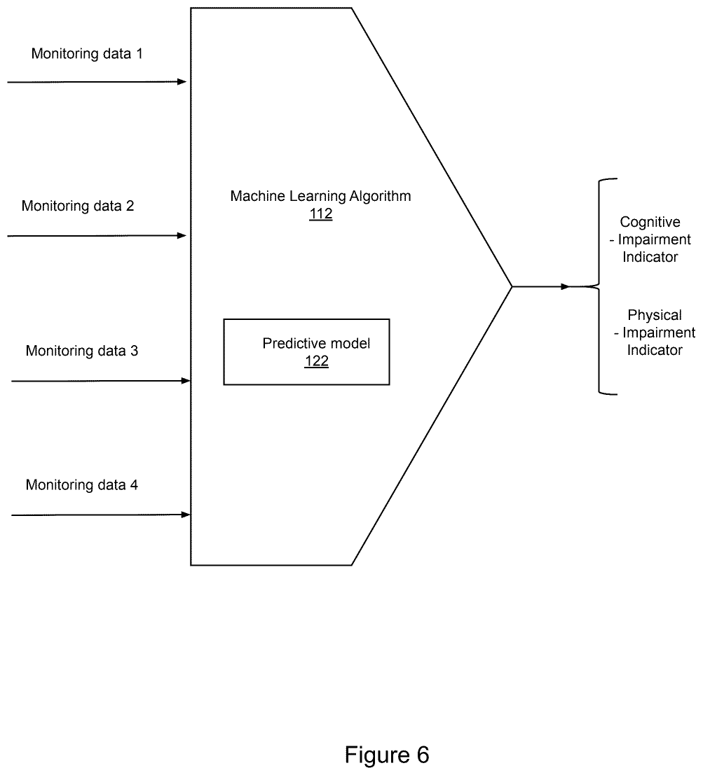

FIG. 6 represents a third implementation with two outputs: the cognitive impairment indicator of FIG. 4 and the physical impairment indicator of FIG. 5. In this case, the predictive model 122 has been generated during the training phase, to provide the capability to determine simultaneously the cognitive impairment and the physical impairment indicators.

For illustration purposes only, FIG. 6 represents four different types of monitoring data being used as inputs. However, any number of inputs greater than one may be used. Furthermore, monitoring data 1 may consist of a single instance or a series of consecutive instances of the same type of monitoring data (the same applies to monitoring data 2, 3 and 5).

The types of monitoring data used as inputs for the implementation illustrated in FIG. 6 may be entirely different of those used for the implementations illustrated in FIGS. 4 and 5, or some of them may be common to those used for the implementations illustrated in FIG. 4 or 5.

Furthermore, the processing unit 110 of the monitoring system 100 may operate in one of the following configurations: execute the implementation of the machine learning algorithm 112 illustrated in FIG. 4 only to determine the cognitive impairment indicator, execute the implementation of the machine learning algorithm 112 illustrated in FIG. 5 only to determine the physical impairment indicator, execute simultaneously the implementation of the machine learning algorithm 112 illustrated in FIG. 4 to determine the cognitive impairment indicator and execute the implementation of the machine learning algorithm 112 illustrated in FIG. 5 to determine the physical impairment indicator, execute the implementation of the machine learning algorithm 112 illustrated in FIG. 6 to simultaneously determine the cognitive impairment indicator and the physical impairment indicator.

The impairment indicator(s) generated by the machine learning algorithm 112 are further processed by the processing unit 110 of the monitoring system 100.

In one exemplary implementation, the impairment indicator(s) are transmitted (via the communication interface 130) to one or more third party devices 300 (only one third party device 300 is represented in FIG. 1 for simplification purposes). Each third party device 300 is owned by a person interested in knowing the current status of the monitored person in terms of cognitive and/or physical impairment (e.g. a member of the family, a close friend, a health worker, etc.). Optionally, the impairment indicator is transmitted only when it is representative of an impairment (it is not transmitted when the indicator suggests that no impairment has been detected).

Referring now concurrently to FIGS. 1 and 7, another exemplary implementation using the impairment indicator(s) is represented in FIG. 7.

The processing unit 110 illustrated in FIG. 7 corresponds to the one illustrated in FIG. 1. The processing unit 110 executes an assistance software 116. The assistance software 116 implements several functionalities for providing assistance in the daily life of the monitored person, such as facilitating the planning and execution of tasks performed by the monitored person, entertaining the monitored person, intellectually stimulating the monitored person, managing and facilitating interactions with individuals (e.g. member(s) of the family, friend(s), health worker(s), etc.) responsible for the well-being of the monitored person, etc.

The activation of a given functionality of the assistance software 116 depends on the level of cognitive and/or physical impairment of the monitored person. Consequently, based at least on the determined value of the impairment indicator (cognitive and/or physical), a determination is made that one or more functionalities of the assistance software 116, which were not currently active, need to be activated.

For example, as mentioned previously, the indicator is one among a pre-defined set of values representatives of different levels of cognitive or physical impairment (e.g. no impairment, light impairment, strong impairment, critical impairment, etc.). For illustration purposes, we consider that the monitored person is initially in the state of light impairment (cognitive or physical). Functionalities of the assistance software 116 corresponding to this state are activated and operate on a regular basis. For a given period of time, the impairment indicator is determined to be in accordance with the light impairment state. At some point, the impairment indicator is determined to be in the state of strong impairment. Additional functionalities of the assistance software 116 corresponding to this new state of impairment are automatically activated and operate on a regular basis.

Alternatively, instead of automatically activating the additional functionalities corresponding to the new state of impairment, a recommendation to activate them (e.g. providing a list of the additional functionalities to be activated) is transmitted to the third party device 300 illustrated in FIG. 1. The user of the third party device 300 takes the decision to activate or not the additional functionalities suggested by the monitoring system 100.

One particular aspect is the initial configuration of the assistance software 116, when it is first deployed to assist the person. A determination needs to be made to select which functionalities of the assistance software 116 need to be activated. At this stage (deployment phase), most of the previously mentioned monitoring data are not available, to be taken into consideration for this determination.

However, the previously described information (monitoring survey related to cognitive and/or physical capabilities of the person, personal information form defining a profile and/or preferences of the person) received from another device 220 can be used for this determination. A machine learning algorithm with a predictive model specifically generated to support the initial configuration of the assistance software 116 can be used. The inputs of the machine learning algorithm comprise at least some of the information provided by the aforementioned monitoring survey and/or personal information form. The outputs of the machine learning algorithm comprise a selection of functionalities of the assistance software 116 to be activated, based on the inputs.

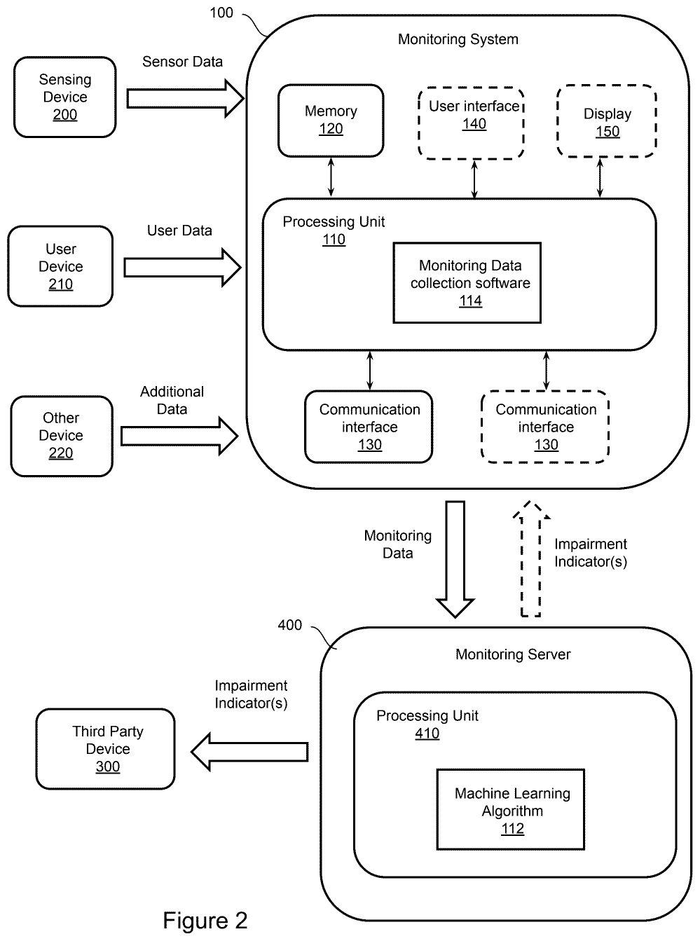

Referring now concurrently to FIGS. 1 and 2, another implementation of the system for performing cognitive or physical impairment monitoring is represented in FIG. 2.

A monitoring server 400 is represented in FIG. 2. The monitoring server 400 comprises a processing unit 410 similar to the processing unit 110 of the monitoring system 100. The monitoring server 400 comprises additional components not represented in FIG. 2 for simplification purposes: memory (similar to the memory 120 of the monitoring system 100), at least one communication interface (similar to the communication 130 of the monitoring system 100), etc.

Instead of having the processing unit 110 of the monitoring system 100 executing the machine learning algorithm 112 as illustrated in FIG. 1, the processing unit 310 of the monitoring server 300 executes the machine learning algorithm 112 in FIG. 2. Thus, the monitoring data generated by the monitoring data collection software 114 are transmitted to the monitoring server 400, to be processed by the machine learning algorithm 112 on the monitoring server 400, to generate the impairment indicator(s) (cognitive and/or physical) as described previously.

As described previously, the monitoring server 400 transmits the impairment indicator(s) to one or more third party devices 300. Optionally, the monitoring server 400 also transmits the impairment indicator(s) to the monitoring system 100.

The previously described determination, based on the value of the impairment indicator, of one or more functionalities of the assistance software 116 (illustrated in FIG. 7), which were not currently active, to be activated can be performed by the monitoring server 400 or the monitoring system 100.

FIG. 2 illustrates a cloud-based architecture, where the monitoring systems 100 deployed at the user premises are only used for collecting monitoring data, generated based on the data transmitted by the devices 200, 210 and 220. The centralized monitoring server 400 is in charge of the processing of the monitoring data collected at a plurality of user premises, to generate the corresponding impairment indicator(s).

In still another implementation not represented in the Figures, no monitoring system 100 is deployed at the user premises. The devices 200, 210 and 220 directly transmit their data to the monitoring server 400. In this case, the processing unit 410 of the monitoring server 400 also executes the monitoring data collection software 114, to generate the monitoring data used by the machine learning algorithm 112, based on the data transmitted by the devices 200, 210 and 220.

As mentioned previously, in this cloud-based architecture, the centralized monitoring server 400 is in charge of the processing of the raw data collected at a plurality of user premises, to generate the monitoring data, and to further generate the corresponding impairment indicator(s).

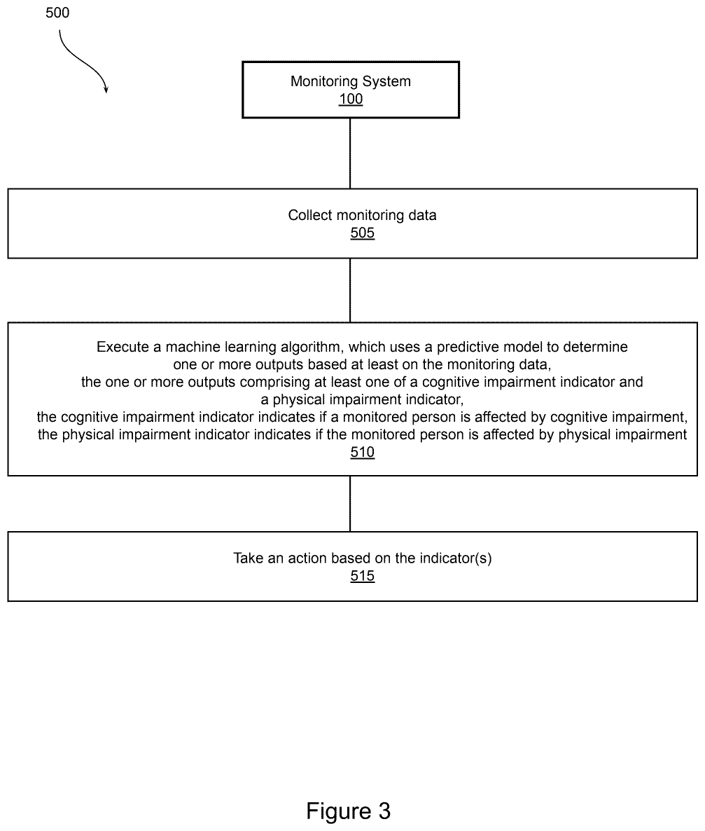

Reference is now made concurrently to FIGS. 1, 2 and 3, where FIG. 3 represents a method 500 using machine learning to monitor cognitive or physical impairment. FIG. 3 represents a monitoring system 100 implementing the steps of the method 500. In a first implementation illustrated in FIG. 1, the monitoring system 100 is the monitoring system 100. In a second implementation illustrated in FIG. 2, the monitoring system 100 is the monitoring server 400. The steps of the method 500 are described generically in FIG. 3, in order to support the first and the second implementations. However, more details will be provided in the following paragraphs for the steps which need to be further adapted to each implementation.

One or more computer program(s) have instructions for implementing at least some of the steps of the method 500. The instructions are comprised in a non-transitory computer readable medium (e.g. memory) of the monitoring system 100. The instructions provide for using machine learning to monitor cognitive or physical impairment, when executed by the processing unit 110 of the monitoring system 100. The instructions are deliverable to the monitoring system 100 via an electronically-readable media such as a storage media (e.g. USB key, etc.), or via communication links (e.g. via a communication network through a communication interface of the monitoring system 100).

The method 500 comprises the step 505 of collecting monitoring data. Step 505 is performed by the processing unit 110 of the monitoring system 100.

In the configuration illustrated in FIG. 1, step 505 is implemented as follows. The processing unit 110 of the monitoring system 100 receives data from a plurality of devices (e.g. sensing device(s) 200, user device(s) 210 and other device(s) 220) located in the living environment of the monitored person or used by individuals visiting the living environment of the monitored person. The processing unit 110 of the monitoring system 100 further generates the monitoring data based on the received data. Step 505 is performed by the monitoring data collection software 114 executed by the processing unit 110.

In the configuration illustrated in FIG. 2, where the monitoring system 100 is the monitoring server 400 (not located in the living environment of the monitored person), step 505 is implemented as follows. The processing unit 410 of the monitoring server 400 receives the monitoring data from the monitoring system 100. The generation of the monitoring data by the monitoring system 100 is performed by the monitoring data collection software 114 as described in the previous paragraph.

In another configuration not illustrated in the Figures, where the monitoring system 100 is the monitoring server 400 (not located in the living environment of the monitored person), step 505 is implemented as follows. The processing unit 410 of the monitoring server 400 directly receives data from a plurality of devices (e.g. sensing device(s) 200, user device(s) 210 and other device(s) 220) located in the living environment of the monitored person. The processing unit 410 of the monitoring server 400 further generates the monitoring data based on the received data. Step 505 is performed by the monitoring data collection software 114 executed by the processing unit 410 of the monitoring server 400 (instead of the processing unit 110 of the monitoring system 100 as illustrated in FIGS. 1 and 2).

Optionally, as mentioned previously, other device(s) 220 not located in the living environment of the monitored person also transmit data, which are used for generating the monitoring data at step 505.

The method 500 comprises the step 510 of executing the machine learning algorithm 112, which uses a predictive model to determine one or more outputs based at least on the monitoring data collected at step 505. The one or more outputs comprises at least one of the cognitive impairment indicator and the physical impairment indicator. Step 510 is performed by the processing unit of the monitoring system 100.

In the configuration illustrated in FIG. 1, where the monitoring system 100 is the monitoring system 100, the machine learning algorithm 112 is executed by the processing unit 110 of the monitoring system 100.

In the configuration illustrated in FIG. 2, where the monitoring system 100 is the monitoring server 400, the machine learning algorithm 112 is executed by the processing unit 410 of the monitoring server 400.

The method 500 comprises the step 515 of taking an action based on the indicator(s) determined at step 510. Depending on which indicator(s) is (are) determined at step 510, the action is taken based on the value of the cognitive impairment indicator only, on the value of the physical impairment indicator only, or the values of the cognitive impairment and physical impairment indicators considered in combination. Step 515 is performed by the processing unit of the monitoring system 100.

A first exemplary action comprises transmitting the indicator(s) to a third party device 300, as illustrated in FIGS. 1 and 2.

In the configuration illustrated in FIG. 2, where the monitoring system 100 is the monitoring server 400, the indicator(s) is (are) also optionally transmitted to the monitoring system 100.

A second exemplary action comprises determining, based on the impairment indicator(s), that one or more functionalities of the assistance software 116 illustrated in FIG. 7 need to be activated. The assistance software 116 and its control based on the impairment indicator(s), has been described previously in relation to FIG. 7.

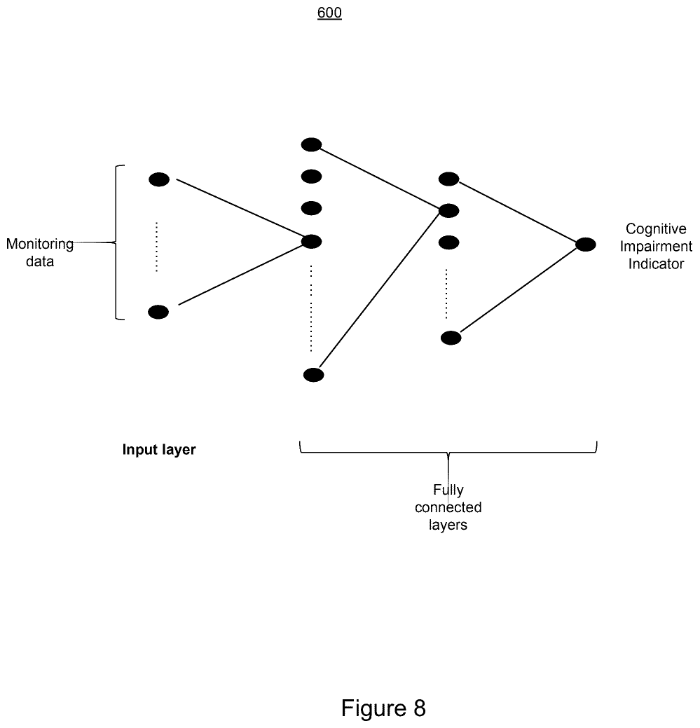

Reference is now made concurrently to FIGS. 4 and 8, where FIG. 8 represents an exemplary implementation of the machine learning algorithm 112 illustrated in FIG. 4 by a neural network 600.

A person skilled in the art would readily adapt FIG. 8 and the teachings of the following paragraphs, to the implementation of the machine learning algorithms 112 illustrated in FIGS. 5 and 6 by a neural network.

The neural network 600 includes an input layer for receiving the inputs, followed by a plurality of fully connected layers. The last layer among the plurality of fully connected layers is an output layer for outputting the output(s). The output(s) are generated by the neural network 600, by applying the predictive model 122 to the inputs.

The neural network 600 represented in FIG. 8 is for illustration purposes only. A person skilled in the art will readily understand that other implementations of the neural network 600 may be used.

The output layer comprises one neuron for outputting the cognitive impairment indicator. The input layer comprises a plurality of neurons for receiving the monitoring data. Any previously described type of monitoring data can be received via one of the neurons of the input layer. Alternatively, several consecutive instances of a previously described type of monitoring data can be received via several corresponding neurons of the input layer.

In an alternative implementation corresponding to FIG. 5 (not represented in FIG. 8), the output layer comprises one neuron for outputting the physical impairment indicator. In another alternative implementation corresponding to FIG. 6 (not represented in FIG. 8), the output layer comprises one neuron for outputting the cognitive impairment indicator and one neuron for outputting the physical impairment indicator.

The operations of the fully connected layers are well known in the art. The number of fully connected layers is an integer greater than 2, including the output layer (FIG. 8 represents three fully connected layers, including the output layer, for illustration purposes only). The number of neurons in each fully connected layer may vary. During the training phase of the neural network, the number of fully connected layers and the number of neurons for each fully connected layer are selected; and may be adapted experimentally.

In an alternative implementation not represented in FIG. 8, the neural network 600 comprises a convolutional layer, optionally followed by a pooling layer, for receiving (instead of the input layer illustrated in FIG. 8) and processing at least some of the monitoring data. The outputs of the convolutional layer and optional pooling layer are further processed by the fully connected layers. For example, the convolutional layer is used when some of the monitoring data are in the form of a matrix, which is processed by the convolutional layer.

Following is a description of a procedure for training the neural network 600 to generate the cognitive impairment indicator. The training procedure is generally implemented by a dedicated training server (not represented in the Figures). The training procedure can be adapted by a person skilled in the art to other types of machine learning algorithms 112.

The training procedure comprises a step of initializing the predictive model 112. The initialization step comprises defining a number of layers of the neural network, a functionality for each layer (e.g. input layer, fully connected layer, etc.), initial values of parameters used for implementing the functionality of each layer, etc. For example, the initialization of the parameters of a fully connected layer includes determining the number of neurons of the fully connected layer and determining an initial value for the weights of each neuron. Different algorithms (well documented in the art) can be used for allocating an initial value to the weights of each neuron. A comprehensive description of the initialization of the predictive model is out of the scope of the present disclosure, since it is well known in the art.

The training procedure comprises a step of generating training data. The training data comprise a plurality of instances of inputs and a corresponding plurality of instances of expected output(s). In the configuration illustrated in FIG. 8, each instance of inputs consists of a set of values for the monitoring data. Each corresponding output consists of an expected value for the cognitive impairment indicator. The set of training data needs to be large enough to properly train the neural network.

The training procedure comprises a step (I) of executing the neural network 600, using the predictive model 122 to generate respective instances of the calculated output based on the instances of inputs of the training data.

The training procedure comprises a step (II) of adjusting the predictive model 122 of the neural network 600, to minimize a difference between the instances of expected output and the corresponding instances of calculated output. For example, for a fully connected layer of the neural network, the adjustment comprises adjusting the weights associated to the neurons of the fully connected layer.

Various algorithms may be used for minimizing the difference between the expected output and the calculated output. For example, the predictive model is adjusted so that a difference between the expected output and the calculated output is lower than a threshold (e.g. a difference of only 1% is tolerated).

At the end of the training procedure, the neural network 600 is considered to be properly trained (the predictive model 122 of the neural network 600 has been adjusted so that a difference between the expected output and the calculated output has been sufficiently minimized). The predictive model 122, comprising the adjusted parameters of the neural network 600 (e.g. the weights), is transmitted to the monitoring system 100 of FIG. 1 or the monitoring server 400 of FIG. 2, to be stored in their respective memory. Test data are optionally used to validate the accuracy of the predictive model 122. The test data are different from the training data used during the training procedure.

Various techniques well known in the art of neural networks can be used for performing step (II). For example, the adjustment of the predictive model 122 of the neural network 600 at step (II) uses back propagation. Other techniques, such as the usage of bias in addition to the weights (bias and weights are generally collectively referred to as weights in the neural network terminology), reinforcement learning, supervised or unsupervised learning, etc., may also be used.

The following is a (non-exhaustive) chart listing exemplary metrics which can be collected, monitored, measured, determined, calculated, etc. The metrics are used as inputs of the machine learning algorithm for determining at least one of a cognitive impairment indicator and a physical impairment indicator.

| Metric | Sensor type | How it measures |
|---|---|---|
| Functional | Motion sensors, smart | Tracks movement patterns, |
| activities | appliances, | appliance use, or task |
| | wearables, radar, | completion (e.g., cooking, |
| | heat sensor, audio | dressing). Detects changes in |
| | sensor | frequency or efficiency of |
| | | daily activities. |
| Sleep | Bed sensors, | Monitors sleep duration, |
| disturbances | wearables, actigraphy | awakenings, and sleep quality |
| | devices, radar, heat | through movement, heart rate, |
| | sensor, audio sensor | or pressure changes. |
| Mobility and | Motion sensors, | Measures walking speed, |
| gait | wearables, pressure | stride variability, or fall |
| | mats, radar | frequency, which correlate |
| | | with executive function and |
| | | cognitive health. |
| Social | Audio sensors, | Detects frequency and duration |
| interaction | smartphones, | of conversations or social |
| | wearables | activities via voice detection |
| | | or phone usage patterns. |
| Cognitive | Smart home systems, | Infers cognitive changes |
| decline | ambient sensors | through anomalies in routine |
| (indirect) | | (e.g., forgetting to turn off |
| | | lights, repeated actions). |
| Neuro- | Wearables, audio | Tracks heart rate variability, |
| psychiatric | sensors, radar | vocal tone, or activity levels |
| symptoms | | to detect mood changes or |
| (e.g. apathy, | | agitation. |
| agitation) | | |
| Medication | Smart pill dispensers, | Monitors whether medications |
| adherence | Radio-Frequency | are taken on schedule, |
| | Identification (RFID) | indicating memory or executive |
| | tags | function issues. |

Although the present disclosure has been described hereinabove by way of non-restrictive, illustrative embodiments thereof, these embodiments may be modified at will within the scope of the appended claims without departing from the spirit and nature of the present disclosure.

What is claimed is:

1. A method using machine learning to monitor cognitive or physical impairment, the method comprising:
receiving by a processing unit of a monitoring system data from a plurality of devices located in a living environment of a monitored person;
generating by the processing unit monitoring data based on the received data; and
executing by the processing unit a machine learning algorithm, the machine learning algorithm using a predictive model to determine one or more outputs based at least on the monitoring data, the one or more outputs comprising at least one of a cognitive impairment indicator and a physical impairment indicator, the cognitive impairment indicator indicating whether the monitored person is affected by cognitive impairment, the physical impairment indicator indicating whether the monitored person is affected by physical impairment.

2. The method of claim 1, wherein the one or more outputs of the machine learning algorithm comprises the cognitive impairment indicator.

3. The method of claim 2, further comprising transmitting the cognitive impairment indicator to a third party device.

4. The method of claim 2, further comprising determining based at least on the cognitive impairment indicator that one or more functionalities of an assistance software need to be activated, the assistance software providing assistance in the daily life of the monitored person.

5. The method of claim 1, wherein the one or more outputs of the machine learning algorithm comprises the physical impairment indicator.

6. The method of claim 5, further comprising transmitting the physical impairment indicator to a third party device.

7. The method of claim 5, further comprising determining based at least on the physical impairment indicator that one or more functionalities of an assistance software need to be activated, the assistance software providing assistance in the daily life of the monitored person.

8. The method of claim 1, wherein the plurality of devices located in the living environment of the monitored person comprise at least one of the following: a sensing device, a personal electronic device and a smart appliance.

9. The method of claim 1, wherein the monitoring data comprise at least one of the following: an occurrence of an activity performed by the monitored person, a duration of an activity performed by the monitored person, a number of occurrences of an activity performed by the monitored person, an occurrence of an interaction of the monitored person with a device or an object located in the living environment of the monitored person, a duration of an interaction of the monitored person with a device or an object located in the living environment of the monitored person, a number of occurrences of an interaction of the monitored person with a device or an object located in the living environment of the monitored person, an occurrence of a fall of the monitored person, a number of occurrences of a fall of the monitored person, an average speed of the monitored person when walking in the living environment, an average time spent in an area, a maximum time spent in an area, a minimum time spent in an area, a number of visits to an area, a sleep quality metric, a health metric, a variation in the value of a metric generated based on the received data, information related to at least one of physical and cognitive capabilities of the monitored person, and personal information related to the monitored person.

10. The method of claim 1, wherein the machine learning algorithm implements a neural network, the predictive model comprising weights of the neural network.

11. A non-transitory computer readable medium comprising instructions executable by a processing unit of a monitoring system, the execution of the instructions by the processing unit of the device providing for using machine learning to monitor cognitive or physical impairment by:
receiving by the processing unit data from a plurality of devices located in a living environment of a monitored person;
generating by the processing unit monitoring data based on the received data; and
executing by the processing unit a machine learning algorithm, the machine learning algorithm using a predictive model to determine one or more outputs based at least on the monitoring data, the one or more outputs comprising at least one of a cognitive impairment indicator and a physical impairment indicator, the cognitive impairment indicator indicating whether the monitored person is affected by cognitive impairment, the physical impairment indicator indicating whether the monitored person is affected by physical impairment.

12. A monitoring system comprising:
at least one communication interface;
memory storing a predictive model; and
a processing unit for:
receiving via the at least one communication interface data from a plurality of devices located in a living environment of a monitored person;
generating monitoring data based on the received data; and
executing a machine learning algorithm, the machine learning algorithm using the predictive model to determine one or more outputs based at least on the monitoring data, the one or more outputs comprising at least one of a cognitive impairment indicator and a physical impairment indicator, the cognitive impairment indicator indicating whether the monitored person is affected by cognitive impairment, the physical impairment indicator indicating whether the monitored person is affected by physical impairment.

13. The monitoring system of claim 12, wherein the one or more outputs of the machine learning algorithm comprises the cognitive impairment indicator.

14. The monitoring system of claim 13, wherein the processing unit further transmits the cognitive impairment indicator to a third party device.

15. The monitoring system of claim 13, wherein the processing unit further determines based at least on the cognitive impairment indicator that one or more functionalities of an assistance software need to be activated, the assistance software providing assistance in the daily life of the monitored person.

16. The monitoring system of claim 12, wherein the one or more outputs of the machine learning algorithm comprises the physical impairment indicator.

17. The monitoring system of claim 16, wherein the processing unit further transmits the physical impairment indicator to a third party device.

18. The monitoring system of claim 16, wherein the processing unit further determines based at least on the physical impairment indicator that one or more functionalities of an assistance software need to be activated, the assistance software providing assistance in the daily life of the monitored person.

19. The monitoring system of claim 12, wherein the plurality of devices located in the living environment of the monitored person comprise at least one of the following: a sensing device, a personal electronic device and a smart appliance.

20. The monitoring system of claim 12, wherein the monitoring data comprise at least one of the following: an occurrence of an activity performed by the monitored person, a duration of an activity performed by the monitored person, a number of occurrences of an activity performed by the monitored person, an occurrence of an interaction of the monitored person with a device or an object located in the living environment of the monitored person, a duration of an interaction of the monitored person with a device or an object located in the living environment of the monitored person, a number of occurrences of an interaction of the monitored person with a device or an object located in the living environment of the monitored person, an occurrence of a fall of the monitored person, a number of occurrences of a fall of the monitored person, an average speed of the monitored person when walking in the living environment, an average time spent in an area, a maximum time spent in an area, a minimum time spent in an area, a number of visits to an area, a sleep quality metric, a health metric, a variation in the value of a metric generated based on the received data, information related to at least one of physical and cognitive capabilities of the monitored person, and personal information related to the monitored person.
